(19) United States
(12) Patent Application Publication
(10) Pub. No.: US 2022/0338477 A1
(43) Pub. Date: Oct. 27, 2022

(54) MYCOBACTERIUM BACTERICIDE

(71) Applicant: SANKEI CO., LTD., Osaka (JP)

(72) Inventor: Hisataka GODA, Osaka (JP)

(21) Appl. No.: 17/763,258

(22) PCT Filed: Sep. 23, 2020
(86) PCT No.: PCT/JP2020/035702

§ 371 Date: Mar. 24, 2022

(30) Foreign Application Priority Data
Sep. 24, 2019 (JP) 2019-173414

(51) Int. Cl. A01N 59/08; A01N 25/02; A61K 33/20; A61P 31/04; C01B 11/08

(52) U.S. Cl. CPC A01N 59/08; A01N 25/02; A61K 33/20; A61P 31/04; C01B 11/08

(57) ABSTRACT

Provided is a mycobacterium bactericide. The mycobacterium bactericide includes chlorous acid water. The free chlorine concentration of the chlorous acid water (as Cl) may be 50 ppm or more. A biofilm may be formed with the mycobacterium. The mycobacterium may be Mycobacterium intracellulare, Mycobacterium avium, Mycolicibacterium fortuitum subsp. fortuitum, Mycobacterium runyonii, Mycobacterium abscessus, Mycobacterium kansasii, M. avium subsp. paratuberculosis, or Mycobacterium ulcerans. A medicine containing chlorous acid water is provided for preventing and/or treating MAC lung disease.

### TECHNICAL FIELD

The present disclosure relates to an agent for use in sterilization of mycobacterium and an agent for use in preventing and/or treating MAC lung disease. The present disclosure also relates to a method of sterilizing a mycobacterium and a method of preventing and/or treating MAC lung disease. The present disclosure also relates to a chlorous acid aqueous solution for use in sterilizing a mycobacterium and a chlorous acid aqueous solution for use in preventing and/or treating MAC lung disease. The present disclosure also relates to an agent for use in sterilization of bacteria that form a biofilm, a method of sterilizing bacteria that form a biofilm, and a chlorous acid aqueous solution for use in sterilizing bacteria that form a biofilm.

### BACKGROUND ART

The prevalence of MAC lung disease has increased and become problematic in developed nations in recent years. Mycobacterium, which is the bacteria causing MAC lung disease, forms a biofilm. Thus, a mycobacterium is known as a bacterium which tends to be resistant to an effect of an agent and survives.

The primary active ingredient of a chlorous acid aqueous solution is chlorous acid (HClO₂). Chlorous acid aqueous solutions have drawn interest as an antimicrobial agent, disinfecting agent, microbe-removing agent, germicidal agent, antiviral agent, and food additive/disinfectant. The inventors have discovered chlorous acid aqueous solutions and methods of manufacture thereof, which have been filed as a patent application after confirming a disinfecting effect on E. coli (Patent Literature 1).

### CITATION LIST

### Patent Literature

[PTL 1] International Publication No. WO 2008/026607

### SUMMARY OF INVENTION

### Solution to Problem

The inventors completed the present disclosure as a result of diligent study by finding that a chlorous acid aqueous solution is effective for sterilizing mycobacteria, and a chlorous acid aqueous solution is effective for sterilizing mycobacteria that form a biofilm. The inventors also completed the present disclosure by finding that a chlorous acid aqueous solution is effective in the prevention/treatment of MAC lung disease.

For example, the present invention provides the following items.

### (Item 1)

An agent for use in sterilization of mycobacterium comprising a chlorous acid aqueous solution.

### (Item 2)

The agent for use in sterilization of item 1, wherein a free chlorine concentration (as Cl) of the chlorous acid aqueous solution is 50 ppm or greater and 60000 ppm or less.

### (Item 3)

The agent for use in sterilization of item 1, wherein a free chlorine concentration (as Cl) of the chlorous acid aqueous solution is 100 ppm or greater and 60000 ppm or less.

### (Item 4)

The agent for use in sterilization of any one of items 1 to 3, wherein the mycobacterium has formed a biofilm.

### (Item 5)

The agent for use in sterilization of any one of items 1 to 4, wherein the mycobacterium is a nontuberculous mycobacterium.

### (Item 6)

The agent for use in sterilization of any one of items 1 to 5, wherein the mycobacterium is Mycobacterium intracellulare, Mycobacterium avium, Mycolicibacterium fortuitum subsp. fortuitum, Mycobacterium runyonii, Mycobacterium abscessus, Mycobacterium kansasii, M. avium subsp. paratuberculosis, or Mycobacterium ulcerans.

### (Item 7)

An agent for use in preventing and/or treating MAC lung disease, comprising a chlorous acid aqueous solution.

### (Item 8)

The agent of item 7, wherein a free chlorine concentration (as Cl) of the chlorous acid aqueous solution is 50 ppm or greater and 60000 ppm or less.

### (Item 9)

The agent of item 7, wherein a free chlorine concentration (as Cl) of the chlorous acid aqueous solution is 100 ppm or greater and 60000 ppm or less.

### (Item 9A)

The agent of any one of items 7 to 9, comprising a feature of any one or more of the preceding items.

### (Item 10)

A method of sterilizing a mycobacterium by using a chlorous acid aqueous solution.

### (Item 11)

The method of item 10, wherein the mycobacterium is contacted with the chlorous acid aqueous solution in the absence of an organic matter.

### (Item 12)

The method of item 11, wherein a free chlorine concentration (as Cl) of the chlorous acid aqueous solution is 50 ppm or greater and 60000 ppm or less.

### (Item 12A)

The method of any one of items 10 to 12, comprising a feature of any one or more of the preceding items.

### (Item 13)

The method of item 10, wherein the mycobacterium is contacted with the chlorous acid aqueous solution in the presence of an organic matter.

### (Item 13A)

The method of claim 13, comprising a feature of any one or more of the preceding items.

### (Item 14)

The method of item 13, wherein a free chlorine concentration (as Cl) of the chlorous acid aqueous solution is 100 ppm or greater and 60000 ppm or less.

### (Item 14A)

The method of item 14, comprising a feature of any one or more of the preceding items.

### (Item 15)

A method of preventing and/or treating MAC lung disease in a subject, comprising administering an effective amount of a chlorous acid aqueous solution to the subject, or contacting an effective amount of a chlorous acid aqueous solution with a mycobacterium that causes MAC lung disease.

### (Item 15A)

The method of item 15, comprising a feature of any one or more of the preceding items.

### (Item 16)

A chlorous acid aqueous solution for use in sterilizing a mycobacterium.

### (Item 16A)

The chlorous acid aqueous solution of item 16, comprising a feature of any one or more of the preceding items.

### (Item 17)

A chlorous acid aqueous solution for use in preventing and/or treating MAC lung disease.

### (Item 17A)

The chlorous acid aqueous solution of item 17, comprising a feature of any one or more of the preceding items.

### (Item 18)

An agent for use in sterilization of a bacterium that forms a biofilm, comprising a chlorous acid aqueous solution.

### (Item 18A)

The agent for use in sterilization of item 18, comprising a feature of any one or more of the preceding items.

### (Item 19)

A method of sterilizing a bacterium that forms a biofilm, comprising contacting an effective amount of a chlorous acid aqueous solution with the bacterium that forms a biofilm.

### (Item 19A)

The method of item 19, comprising a feature of any one or more of the preceding items.

### (Item 20)

A chlorous acid aqueous solution for use in sterilizing a bacterium that forms a biofilm.

### (Item 20A)

The chlorous acid aqueous solution of item 20, comprising a feature of any one or more of the preceding items.

### (Item 21)

Use of a chlorous acid aqueous solution in the manufacture of an agent for sterilizing a mycobacterium.

### (Item 21A)

The use of item 21, comprising a feature of any one or more of the preceding items.

### (Item 22)

Use of a chlorous acid aqueous solution in the manufacture of a drug for preventing and/or treating MAC lung disease.

### (Item 22A)

The use of item 221, comprising a feature of any one or more of the preceding items.

### (Item 23)

Use of a chlorous acid aqueous solution in a drug for sterilizing a bacterium that forms a biofilm.

### (Item 23A)

The use of item 23, comprising a feature of any one or more of the preceding items.

The present disclosure is intended so that one or more of the features described above can be provided not only as the explicitly disclosed combinations, but also as other combinations. Additional embodiments and advantages of the present disclosure are recognized by those skilled in the art by reading and understanding the following detailed description as needed.

### Advantageous Effects of Invention

The present disclosure can readily sterilize mycobacteria. The present disclosure can even sterilize mycobacteria that have formed a biofilm.

### BRIEF DESCRIPTION OF DRAWINGS

FIG. 1 shows a test for studying the disinfecting effect of a chlorous acid aqueous solution and sodium hypochlorite on M. intracellulare. A chlorous acid aqueous solution was found to have a disinfecting effect at a free chlorine concentration (as Cl) of 50 ppm in the absence of an organic matter. In the presence of an organic matter (0.5% BSA), a disinfecting effect was found at a free chlorine concentration (as Cl) of 100 ppm. A disinfecting effect was not found for sodium hypochlorite.

FIG. 2 shows a test for studying a disinfecting effect on M. intracellulare that has formed a biofilm. It is shown that viable bacteria were mostly eliminated when treated with a chlorous acid aqueous solution.

FIG. 3 shows observation of the M. intracellulare surface after disinfection by using a scanning electron microscope (SEM). FIG. 3 shows before treatment (left) and after treatment with sterilized water for 30 minutes (right).

FIG. 4 shows observation of the M. intracellulare surface after disinfection by using a scanning electron microscope (SEM). FIG. 4 shows before treatment (left) and after treatment with sodium hypochlorite at a free chlorine concentration of 200 ppm for 30 minutes (right).

FIG. 5 shows observation of the M. intracellulare surface after disinfection by using a scanning electron microscope (SEM). FIG. 5 shows before treatment (left) and after treatment with a chlorous acid aqueous solution at a free chlorine concentration of 200 (386) ppm or greater for 30 minutes (right).

FIG. 6 shows results of pulsed-field gel electrophoresis of DNA after disinfection of M. intracellulare. It can be understood that a band is formed at the same position as water when treated with sodium hypochlorite, but a band is eliminated and DNA is shredded when treated with a chlorous acid aqueous solution.

FIG. 7 shows results of studying the disinfecting effect in the absence of an organic matter on various mycobacteria. The bacteria are, in order from the left, JCM135429, JCM135430, JCM135431, JCM135432, JCM6387, JCM6390, and JCM13569 at each concentration.

FIG. 8 shows results of studying the disinfecting effect in the presence of an organic matter (0.5% BSA) on various mycobacteria. The bacteria are, in order from the left, JCM135429, JCM135430, JCM135431, JCM135432, JCM6387, JCM6390, and JCM13569 at each concentration.

### DESCRIPTION OF EMBODIMENTS

The present disclosure is described hereinafter in more detail. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the present disclosure pertains. In case of a contradiction, the present specification (including the definitions) takes precedence.

The abbreviations used herein have the conventional meaning within the scope of the art unless specifically noted otherwise.

The term “about” for a value or parameter herein includes variations about the value or parameter itself. Unless specifically noted otherwise, “about X” for example includes “X” itself as well as values with an acceptable error of ±10% therefrom.

As used herein, “chlorous acid aqueous solution” refers to an aqueous solution comprising chlorous acid (HClO₂) used as a disinfecting agent, which can stably sustain chlorous acid (HClO₂) over a long period of time. The presence of a chlorous acid aqueous solution can be confirmed if an absorbent section comprising an acidic chlorite ion (H⁺+ClO₂⁻) representing a peak near 260 nm and an absorbent section comprising chlorine dioxide (ClO₂) representing a peak near 350 nm can be simultaneously observed between wavelengths of 240 to 420 nm in the UV spectrum, i.e., if a double peak is exhibited, when a sample of chlorous acid aqueous solution is measured with a spectrophotometer.

Chlorous acid aqueous solutions can be prepared by the methods disclosed in International Publication No. WO 2008/026607, WO 2014/188310, WO 2014/188311, WO 2014/188312, WO 2015/093062, and WO 2017/170904.

“Chlorous acid aqueous solution” is a disinfectant that was designed as a food additive on Feb. 1, 2013 and has chlorous acid (HClO₂) as the primary active ingredient. The primary active ingredient chlorous acid (HClO₂) of such a “chlorous acid aqueous solution” is a semi-stable chemical substance, which is approved by the USDA and FDA as a food additive: processing aid as an especially safe substance. The chlorous acid aqueous solutions used in the present disclosure which fall under a food additive, and those that do not fall under a food additive, can be used, and can be provided as a pharmaceutical product, quasi-drug, or various other products.

Moreover, “chlorous acid aqueous solution” can exert a potent disinfecting effect even in the presence of an organic matter. Chlorous acid aqueous solutions are highly praised, i.e., “only chlorous acid aqueous solution was capable of inactivation to the detection limit or below under all load conditions”, in “Heisei 27 Nendo Norouirusu no Fukatsuka Joken ni Kansuru Chosa [2015 Investigation on Norovirus Inactivation Conditions]” at the National Institute of Health Sciences (NIHS). With the revisions of the Ordinance for Enforcement of the Food Sanitation Act, listing of chlorous acid aqueous solution is ongoing in order of incidents of large scale food poisoning such as “Tairyo Chori Shisetsu Chori Manyuaru [Manual for Food Preparation at Large-scale Food Preparation Facilities]” and “Tsukemono no Eisei Kihan [Code of Hygienic Practice for Pickles]”.

A chlorous acid aqueous solution can also be applied as a pharmaceutical product as a class II germicidal disinfectant. Chlorous acid aqueous solutions were approved as a pharmaceutical product of a class II germicidal disinfectant in 2019 with scheduled sale in 2020. For example, chlorous acid aqueous solutions are added to guidelines such as “Hoikusho ni okeru Kansen Taisaku Gaidorain [Guidelines for Infection Control Measures at Nursery Center]” under administration of the Ministry of Health, Labour and Welfare including “Norouirusu ni Kansuru Q&A [Q&A regarding noroviruses]”, relevant manuals such as “Koreisha Kaigo Shien Shisetsu ni okeru Kansen Taisaku Manyuaru [Manual for Controlling Infection at Elderly Care Support Facility]”, various sanitation codes such as “Bento/Sozai no Eisei Kihan [Code of Sanitation for Bento/Dishes]”, etc., which are also being revised as needed. A chlorous acid aqueous solution is a substance that is supplied to wide ranging markets in food sanitation and environmental sanitation markets in Japan.

“Chlorous acid aqueous solution” with chlorous acid as the primary active ingredient possesses a disinfecting power that is equivalent to or greater than that of other chlorine based agents, i.e., “hypochlorous acid water” and “sodium hypochlorite”. “Chlorous acid aqueous solution” with a characteristic of having gradual reactivity with no instantaneous disinfecting effect (immediate effect), but having a gradual reactivity while having a precise disinfecting power, as well as sustained stable disinfecting power, can exert a disinfecting effect that is precise and accurate, albeit slowly, under a contaminated environment with many organic matters, which had been considered as the most challenging for a chlorine oxide based agents (disinfecting power against microorganisms that are latent in contamination).

For this reason, chlorous acid aqueous solutions can exert an inactivation effect on resistant bacteria that had been difficult to disinfect in the past (heat resistant bacteria with increased resistance through forming spores, drug resistant bacteria on which antibiotics are no longer effective, etc.), fungi such as mold and yeast, and viruses (including non-enveloped viruses). “Chlorous acid aqueous solution” does not require preparation upon use or a dedicated generator or the like. A chlorous acid aqueous solution is safe and can be used anywhere by anyone whenever use thereof is desirable.

In addition, the effect of “chlorous acid aqueous solution” in the presence of an organic matter is listed in “Heisei 27 Nendo Norouirusu no Fukatsuka Joken ni Kansuru Chosa Hokokusho [2015 Investigative Report on Norovirus Inactivation Conditions] (National Institute of Health Sciences, Division of Biomedical Food Research)” on the website of the Ministry of Health, Labour and Welfare.

“Chlorous acid aqueous solution” does not need to be prepared upon use or require a dedicated generator or the like. A chlorous acid aqueous solution is safe and can be used anywhere by anyone whenever use thereof is desirable.

As used herein, “chlorous acid aqueous solution formulation” refers to a formulation that is prepared by using a chlorous acid aqueous solution as an active pharmaceutical ingredient. The additional component, pH, chlorous acid content, free chlorine concentration, etc. can be adjusted depending on the application.

As used herein, “active pharmaceutical ingredient chlorous acid aqueous solution” refers to a chlorous acid aqueous solution used as an active pharmaceutical ingredient. A chlorous acid aqueous solution can be prepared by a method described herein.

Since a “chlorous acid aqueous solution formulation” used in the present disclosure has low stimulation even when directly contacted with an animal, the formulation can be a formulation that does not cause any damage. A chlorous acid aqueous solution formulation used in the present disclosure can be a formulation that does not result in a skin corrosive reaction, erythema, or edema when applied to the skin, maintains normal cornea, iris, and conjunctiva when applied to the eye, and does not exhibit skin sensitivity.

As used herein, “in the absence of an organic matter” refers to substantial absence (including at or below a threshold value) in addition to complete absence of an organic matter, referring to any “state in the absence of an organic matter”.

As used herein, “free chlorine”, “free chlorine concentration”, or “free residual chlorine concentration” is a value that is measured according to Appendix 3 in “Testing method for free residual chlorine and bound chlorine specified by the Minister of Health, Labour and Welfare based on the stipulation of Article 17(2) of the regulation of the Water Supply Act” (hereinafter, colorimetry (DPD indicator)), and is a value obtained by oxidation of a DPD indicator.

As used herein, “nontuberculous mycobacteriosis” refers to an infection by a nontuberculous mycobacterium excluding Mycobacterium tuberculosis and mycobacterium leprae. This is also known as atypical mycobacteriosis. Examples of primary causative bacteria include Mycobacterium avium, Mycobacterium intracellulare, Mycobacterium kansasii, Mycobacterium shinjukuense, and the like.

As used herein, “MAC lung disease” is a Mycobacterium avium complex lung disease, referring to nontuberculous mycobacteriosis that has infected the lung.

### Preferred Embodiments

Preferred embodiments of the present disclosure are described below. Embodiments provided below are provided to facilitate the understanding of the present disclosure. It is understood that the scope of the present disclosure should not be limited to the following descriptions. Thus, it is apparent that those skilled in the art can make appropriate modifications within the scope of the present disclosure by referring to the descriptions herein. It is understood that the following embodiments can be used alone or in combination.

(An Agent for Use in Sterilization of Mycobacterium)

In one aspect of the present disclosure, an agent for use in sterilization of Mycobacterium comprising a chlorous acid aqueous solution is provided.

The free chlorine concentration (as Cl) of the chlorous acid aqueous solution in the agent for use in sterilization of the present disclosure can be at least 50 ppm. The free chlorine concentration (as Cl) of the chlorous acid aqueous solution in the agent for use in sterilization of the present disclosure can be at least 100 ppm. The free chlorine concentration (as Cl) of the chlorous acid aqueous solution in the agent for use in sterilization of the present disclosure can be 50 ppm, 100 ppm, 150 ppm, 200 ppm, 250 ppm, 300 ppm, 350 ppm, 400 ppm, 450 ppm, 500 ppm, 600 ppm, 700 ppm, 800 ppm, 900 ppm, 1000 ppm, or greater, and 60000 ppm, 50000 ppm, 40000 ppm, 30000 ppm, 20000 ppm, 10000 ppm, 5000 ppm, or less. The free chlorine concentration (as Cl) can be any value between such values.

The agent for use in sterilization of the present disclosure can be used when the mycobacterium has formed a biofilm. Examples of the mycobacterium that forms a biofilm include Mycobacterium intracellulare.

The mycobacterium can be a nontuberculous mycobacterium in the agent for use in sterilization of the present disclosure.

The mycobacterium can be Mycobacterium intracellulare, Mycobacterium avium, Mycolicibacterium fortuitum subsp. fortuitum, Mycobacterium runyonii, Mycobacterium abscessus, Mycobacterium kansasii, M. avium subsp. paratuberculosis, or Mycobacterium ulcerans in the agent for use in sterilization of the present disclosure.

(Sterilization of Biofilm Forming Bacteria)

In one aspect, the present disclosure provides an agent and a method for use in sterilizing a bacterium that forms a biofilm.

The free chlorine concentration (as Cl) of a chlorous acid aqueous solution in the agent for use in sterilization of a biofilm forming bacterium of the present disclosure can be at least 50 ppm. The free chlorine concentration (as Cl) of the chlorous acid aqueous solution in the agent for use in sterilization of the present disclosure can be at least 100 ppm. The free chlorine concentration (as Cl) of the chlorous acid aqueous solution in the agent for use in sterilization of the present disclosure can be 50 ppm, 100 ppm, 150 ppm, 200 ppm, 250 ppm, 300 ppm, 350 ppm, 400 ppm, 450 ppm, 500 ppm, 600 ppm, 700 ppm, 800 ppm, 900 ppm, 1000 ppm, or greater, and 60000 ppm, 50000 ppm, 40000 ppm, 30000 ppm, 20000 ppm, 10000 ppm, 5000 ppm, or less. The free chlorine concentration (as Cl) can be any value between such values.

Examples of bacteria that form a biofilm in the agent for use in sterilization of the present disclosure include Mycobacterium (e.g., Mycobacterium intracellulare, etc.), Pseudomonas (e.g., P. aeruginosa, etc.), Staphylococcus, Streptococcus (e.g., S. mutans, etc.), Legionella, Helicobacter, and Mycobacterium.

A bacterium that forms a biofilm in the agent for use in sterilization of the present disclosure can be a nontuberculous mycobacterium.

A bacterium that forms a biofilm in the agent for use in sterilization of the present disclosure can be mycobacterium (e.g., Mycobacterium intracellulare, etc.).

(A Method of Sterilizing Bacteria)

In one aspect of the present disclosure, a method of sterilizing a mycobacterium or a bacterium that forms a biofilm by using a chlorous acid aqueous solution is provided.

The method of the present disclosure can be performed in the absence of an organic matter. The free chlorine concentration (as Cl) of the chlorous acid aqueous solution can be at least 50 ppm in this method. The free chlorine concentration (as Cl) of the chlorous acid aqueous solution can be 50 ppm, 100 ppm, 150 ppm, 200 ppm, 250 ppm, 300 ppm, 350 ppm, 400 ppm, 450 ppm, 500 ppm, 600 ppm, 700 ppm, 800 ppm, 900 ppm, 1000 ppm, or greater, and 60000 ppm, 50000 ppm, 40000 ppm, 30000 ppm, 20000 ppm, 10000 ppm, 5000 ppm, or less. The free chlorine concentration (as Cl) can be any value between such values.

The method of the present disclosure can be performed in the presence of an organic matter. The free chlorine concentration (as Cl) of the chlorous acid aqueous solution can be at least 100 ppm in this method. The free chlorine concentration (as Cl) of the chlorous acid aqueous solution can be 100 ppm, 150 ppm, 200 ppm, 250 ppm, 300 ppm, 350 ppm, 400 ppm, 450 ppm, 500 ppm, 600 ppm, 700 ppm, 800 ppm, 900 ppm, 1000 ppm, or greater, and 60000 ppm, 50000 ppm, 40000 ppm, 30000 ppm, 20000 ppm, 10000 ppm, 5000 ppm, or less. The free chlorine concentration (as Cl) can be any value between such values.

(Treatment/Prevention of MAC Lung Disease)

In one aspect of the present disclosure, an agent for use in preventing and/or treating MAC lung disease comprising a chlorous acid aqueous solution is provided.

The free chlorine concentration (as Cl) of the chlorous acid aqueous solution in the agent of the present disclosure can be at least 50 ppm. The free chlorine concentration (as Cl) of the chlorous acid aqueous solution in the agent of the present disclosure can be at least 100 ppm. The free chlorine concentration (as Cl) of the chlorous acid aqueous solution in the agent of the present disclosure can be 50 ppm, 100 ppm, 150 ppm, 200 ppm, 250 ppm, 300 ppm, 350 ppm, 400 ppm, 450 ppm, 500 ppm, 600 ppm, 700 ppm, 800 ppm, 900 ppm, 1000 ppm, or greater, and 60000 ppm, 50000 ppm, 40000 ppm, 30000 ppm, 20000 ppm, 10000 ppm, 5000 ppm, or less. The free chlorine concentration (as Cl) can be any value between such values.

In one aspect of the present disclosure, a method of preventing and/or treating MAC lung disease in a subject, comprising administering an effective amount of a chlorous acid aqueous solution to the subject, or contacting an effective amount of a chlorous acid aqueous solution with a mycobacterium, is provided.

(Chlorous Acid Aqueous Solution and Manufacturing Example Thereof)

The chlorous acid aqueous solution used in the present disclosure has the features found by the inventors. A chlorous acid aqueous solution manufactured by any method, such as a known manufacturing method described in a reference described above, can be used. Examples of typical composition that can be used include, but are not limited to, a combination of 61.40% chlorous acid aqueous solution, 1.00% potassium dihydrogen phosphate, 0.10% potassium hydroxide, and 37.50% purified water (sold by the Applicant; 72% chlorous acid aqueous solution corresponds to 30000 ppm of chlorous acid). This agent reduces the decrease in chlorous acid due to contact with an organic matter under an acidic condition, but maintains the disinfecting effect. The agent also has a feature of having very low chlorine gas generation, which suppresses amplification of odor of the combination of chlorine and organic matter.

In one embodiment, the chlorous acid aqueous solution of the present disclosure can be produced by adding and reacting sulfuric acid or an aqueous solution thereof to an aqueous sodium chlorate solution at an amount and concentration where the pH value of the aqueous solution can be maintained at 3.4 or lower to generate chloric acid, and subsequently adding hydrogen peroxide in an amount equivalent to or greater than the amount required for a reduction reaction of the chloric acid.

Further, in another embodiment, the chlorous acid aqueous solution of the present disclosure can be produced from adding one compound from inorganic acids and inorganic acid salts, two or more types of one compound, or a combination thereof to an aqueous solution prepared from producing chlorous acid by adding and reacting sulfuric acid or an aqueous solution thereof to an aqueous sodium chlorate solution at an amount and concentration where the pH value of the aqueous solution can be maintained at 3.4 or lower to generate chloric acid, and subsequently adding hydrogen peroxide in an amount equivalent to or greater than the amount required for a reduction reaction of the chloric acid, and adjusting the pH value within the range from 2.9 to 8.5.

Furthermore, in another embodiment, the chlorous acid aqueous solution of the present disclosure can be produced from adding one compound from inorganic acids and inorganic acid salts and organic acids and organic acid salts, two or more types of one compound, or a combination thereof to an aqueous solution prepared from producing chlorous acid by adding and reacting sulfuric acid or an aqueous solution thereof to an aqueous sodium chlorate solution at an amount and concentration where the pH value of the aqueous solution can be maintained at 3.4 or lower to generate chloric acid, and subsequently adding hydrogen peroxide in an amount equivalent to or greater than the amount required for a reduction reaction of the chloric acid, and adjusting the pH value within the range from 3.2 to 8.5.

Further still, in another embodiment, the chlorous acid aqueous solution of the present disclosure can be produced by adding one compound from inorganic acids, inorganic acid salts, organic acids and organic acid salts, two or more types of said one compound, or a combination thereof after adding one compound from inorganic acids and inorganic acid salts, two or more types of one compound, or a combination thereof to an aqueous solution prepared from producing chlorous acid by adding and reacting sulfuric acid or an aqueous solution thereof to an aqueous sodium chlorate solution at an amount and concentration where the pH value of the aqueous solution can be maintained at 3.4 or lower to generate chloric acid, and subsequently adding hydrogen peroxide in an amount equivalent to or greater than the amount required for a reduction reaction of the chloric acid, and adjusting the pH value within the range from 3.2 to 8.5.

Further, in another embodiment, carbonic acid, phosphoric acid, boric acid, or sulfuric acid can be used as the inorganic acid in the method described above.

Further still, in another embodiment, carbonate, inorganic hydroxide, phosphate, or borate can be used as the inorganic acid salt.

Further, in another embodiment, sodium carbonate, potassium carbonate, sodium bicarbonate, or potassium bicarbonate can be used as the carbonate.

Furthermore, in another embodiment, sodium hydroxide, potassium hydroxide, calcium hydroxide, or barium hydroxide can be used as the inorganic hydroxide.

Further still, in another embodiment, disodium hydrogen phosphate, sodium dihydrogen phosphate, trisodium phosphate, tripotassium phosphate, dipotassium hydrogen phosphate, or potassium dihydrogen phosphate can be used as the phosphate.

Further, in another embodiment, sodium borate or potassium borate can be used as the borate.

Furthermore, in another embodiment, succinic acid, citric acid, malic acid, acetic acid, or lactic acid can be used as the organic acid.

Further still, in another embodiment, sodium succinate, potassium succinate, sodium citrate, potassium citrate, sodium malate, potassium malate, sodium acetate, potassium acetate, sodium lactate, potassium lactate, or calcium lactate can be used as the organic acid salt.

In a method of manufacturing an aqueous solution comprising chlorous acid (HClO₂) that can be used as an agent for use in sterilization of bacteria (chlorous acid aqueous solution), chlorous acid (HClO₂) is produced by adding hydrogen peroxide (H₂O₂) in an amount required to produce chlorous acid by a reducing reaction of chloric acid (HClO₃) obtained by adding sulfuric acid (H₂SO₄) or an aqueous solution thereof to an aqueous solution of sodium chlorate (NaClO₃) so that the aqueous solution is in an acidic condition. The basic chemical reaction of this method of manufacturing is represented by the following formula A and formula B.

[Chemical Formula 1]

2NaClO₃+H₂SO₄→2HClO₃+Na₂SO₄↓ (formula A)

HClO₈+H₂O₂→HClO₂+H₂O+O₂↑ (formula A)

Formula A indicates that chloric acid is obtained by adding sulfuric acid (H₂SO₄) or an aqueous solution thereof at an amount and concentration where the pH value of an aqueous sodium chlorate (NaClO₃) solution remains acidic. Next, formula B indicates that chloric acid (HClO₃) is reduced by hydrogen peroxide (H₂O₂) to produce chlorous acid (HClO₂).

[Chemical Formula 2]

HClO₃+H₂O₂→2ClO₂+H₂O+O₂↑ (formula c

2ClO₂+H₂O₂→2HClO₂+O₂↑ (formula D)

2ClO₂+H₂O ⇔HClO₂+HClO₃ (formula E)

2HClO₂⇔H₂O+Cl₂O₃ (formula F)

At this time, chlorine dioxide gas (ClO₂) is generated (formula C). However, coexistence with hydrogen peroxide (H₂O₂) produces chlorous acid (HClO₂) through the reactions in formulas D to F.

Meanwhile, the produced chlorous acid (HClO₂) has a property of being decomposed early into chlorine dioxide gas or chlorine gas due to the presence of chloride ion (Cl⁻), hypochlorous acid (HClO), and other reduction product, or resulting in a decomposition reaction among a plurality of chlorous acid molecules with one another. Thus, it is necessary to be prepared so that chlorous acid (HClO₂) can be sustained for an extended period of time in order to be useful as an agent for use in sterilization of bacteria.

In this regard, chlorous acid (HClO₂) can be stably sustained over an extended period of time from creating a transition state to delay a decomposition reaction by adding one compound from inorganic acids, inorganic acid salts, organic acids, and organic acid salts, two or more types of said one compound, or a combination thereof to the chlorous acid (HClO₂) or chlorine dioxide gas (ClO₂) obtained by the method described above or an aqueous solution containing them.

In one embodiment, it is possible to utilize a mixture prepared from adding one compound from inorganic acids and inorganic acid salts, specifically carbonate or inorganic hydroxides, two or more types of one compound, or a combination thereof to the chlorous acid (HClO₂) or chlorine dioxide gas (ClO₂) obtained by the method described above or an aqueous solution containing them.

In another embodiment, it is possible to utilize a mixture prepared by adding one compound from inorganic acids, inorganic acid salts, organic acids, and organic acid salts, two or more types of one compound, or a combination thereof to an aqueous solution to which one compound from inorganic acids and inorganic acid salts, specifically carbonate or inorganic hydroxide, two or more types of one compound, or a combination thereof is added.

Additionally, in another embodiment, it is possible to utilize a mixture prepared by adding one compound from inorganic acids, inorganic acid salts, organic acids, and organic acid salts, two or more types of one compound, or a combination thereof to the aqueous solution manufactured by the method described above.

Examples of the inorganic acid described above include carbonic acid, phosphoric acid, boric acid, and sulfuric acid. Examples of the inorganic acid salt include phosphate and borate, in addition to carbonate and inorganic hydroxide. Specifically, sodium carbonate, potassium carbonate, sodium bicarbonate, or potassium bicarbonate works well as the carbonate; sodium hydroxide, potassium hydroxide, calcium hydroxide, or barium hydroxide works well as the inorganic hydroxide; disodium hydrogen phosphate, sodium dihydrogen phosphate, trisodium phosphate, tripotassium phosphate, dipotassium hydrogen phosphate, or potassium dihydrogen phosphate works well as the phosphate; and sodium borate or potassium borate works well as the borate. Examples of the organic acid include succinic acid, citric acid, malic acid, acetic acid, and lactic acid. Further, sodium succinate, potassium succinate, sodium citrate, potassium citrate, sodium malate, potassium malate, sodium acetate, potassium acetate, sodium lactate, potassium lactate, or calcium lactate is suitable as the organic acid salt.

When an acid and/or a salt thereof is added, a transition state, such as Na⁺+ClO₂<->Na—ClO₂, K⁺+ClO₂⁻<->K—ClO₂, or H⁺+ClO₂⁻<->H—ClO₂ can be temporarily created to delay the formation of chlorine dioxide (ClO₂) from chlorous acid (HClO₂), which enables the manufacture of an aqueous solution comprising chlorous acid (HClO₂) that sustains chlorous acid (HClO₂) for an extended period of time and generates a low amount of chlorine dioxide (ClO₂).

The following represents the decomposition of chlorite in an acidic solution.

[Chemical Formula 3]

5ClO₂⁻+4H⁺→4ClO₃+5Cl⁻+2H₂O (a)

(5NaClO₂+4CH₃COOH→

4ClO₃+4CH₃COONa+NaCL+2H₂O)

3ClO₂⁻→2ClO₃⁻+Cl⁻ (b)

(3NaClO₃→2NaClO₃+NaCl)autodegradation

ClO₂⁻→CL⁻+2O (c)

As expressed in the formula, the rate of decomposition of an aqueous chlorite solution is greater when pH is lower, i.e., more acidic. Specifically, the absolute rates of reactions (a), (b), and (c) in the formula described above increase. For example, although the ratio accounted for by reaction (a) decreases when pH is lower, the total decomposition rate changes significantly, i.e., to a larger value. Thus, the amount of generated chlorine dioxide (ClO₂) also increases as the pH decreases. Therefore, a lower pH value results in sooner disinfection or bleaching. However, irritable and harmful chlorine dioxide gas (ClO₂) renders an operation more difficult and negatively affects the health of a human being. Further, a reaction of chlorous acid to form chlorine dioxide progresses quicker to render chlorous acid unstable. In addition, the period during which a disinfecting power can be sustained is very short.

When the inorganic acids, inorganic acid salts, organic acids, or organic acid salts described above are added to an aqueous solution comprising chlorous acid (HClO₂), pH values are adjusted in the range of 2.9 to 8.5 from the viewpoint of balancing suppression of the generation of chlorine dioxide and disinfecting power.

(Problems in Comparing/Evaluating Antimicrobial Effects of Chlorous Acid Aqueous Solutions and Sodium Hypochlorite)

Comparison/evaluation of antimicrobial effects of chlorous acid aqueous solutions and sodium hypochlorite is problematic in that the concentration of chlorine oxide can be a description of available chlorine concentration or free chlorine, while antimicrobial effects are dependent on free chlorine, which is the source of oxidation power. In addition, sodium hypochlorite has a near 1:1 relationship between free chlorine and available chlorine concentration, but the available chlorine concentration and free chlorine do not necessarily match for chlorous acid aqueous solutions in the same manner as sodium hypochlorite. For this reason, it is necessary to compare disinfecting powers of both agents on a level field by using oxidation power that represents the antimicrobial effect, i.e., free chlorine, instead of available chlorine concentration.

Oxidation power of a chlorine oxide agent is generally found through a measurement method utilizing colorimetry such as a TMB method or DPD method. However, there is no standard for measuring free chlorine in a chlorous acid aqueous solution in the same manner as sodium hypochlorite. For this reason, calibration curves are created by using 1 mg/L of free chlorine (as Cl) of sodium hypochlorite as 1 oxidation power. Oxidation power can be represented by free chlorine (as Cl). For comparison at the same free chlorine, free chlorine (as Cl) of sodium hypochlorite is generated from Cl radicals, but free chlorine of chlorous acid aqueous solutions has HClO₂ as the generation source. Thus, it is difficult to compare when evaluating with respect to a standard in accordance therewith. Thus, a chlorous acid aqueous solution is compared and evaluated with sodium hypochlorite on a level field by using the same standard through calculation at oxidation power 1=free chlorine (as Cl) of 1 mg/L in the same manner as sodium hypochlorite.

As a measuring method of free chlorine (as Cl), a buffer and a DPD indicator are added to a sample and a wavelength of 510 nm is measured with an absorption spectrophotometer, and for measurement of free chlorine (as Cl) in the presence of an organic matter, measurement is taken at a wavelength of 655 nm using a TMB reagent to find the concentration from the measurement values. Further, a method of conducting a test for studying the disinfecting effect prepares free chlorine (as Cl) of a test agent by a DPD method, contacts each agent with an organic matter-containing bacterial solution, neutralizes the solution with sodium thiosulfate after a certain period of time has passed, and checks the viable bacteria count of the neutral solution.

(Method of Using Agent for Use in Sterilization/Agent of the Present Disclosure)

The agent for use in sterilization/agent of the present disclosure can be used in environmental maintenance (infection control measures) at a hospital, etc. The agents can be directly applied to facilities such as floors, walls, and doors, medical equipment, etc. The agents can also be directly applied to animals including humans.

Reference literatures such as scientific literatures, patents, and patent applications cited herein are incorporated herein by reference to the same extent that the entirety of each document is specifically described.

As described above, the present disclosure has been described while showing preferred embodiments to facilitate understanding. The present disclosure is described hereinafter based on Examples. The above descriptions and the following Examples are not provided to limit the present invention, but for the sole purpose of exemplification. Thus, the scope of the present invention is not limited to the embodiments and Examples specifically described herein and is limited only by the scope of claims.

### Examples

(Quantification Method of Chlorous Acid Aqueous Solution)

About 5 g of the present product is precisely measured. Water is added thereto so that the solution is exactly 100 ml. After 20 ml of the sample solution is accurately measured, placed in an iodine flask, and combined with 10 ml of sulfuric acid (1-10), 1 g of potassium iodide is added thereto. The flask is immediately sealed and shaken thoroughly. A potassium iodide reagent is poured into the top portion of the iodine flask and left standing in the dark for 15 minutes. The plug is then loosened to pour in a potassium iodide reagent and the flask is sealed immediately. After thoroughly shaking and mixing, freed iodine is titrated with 0.1 mol/L sodium thiosulfate (indicator: starch reagent). The indicator is added after the color of the solution has changed to a light yellow color. A blank test is separately conducted for correction (1 mL of 0.1 mol/L sodium thiosulfate solution=1.711 mg of HClO₂).

### Manufacturing Examples

Chlorous acid aqueous solution formulations used in the following Examples were manufactured as follows. Chlorous acid aqueous solution may be abbreviated as “CAAS” herein, but the terms are synonymous.

Component Analysis Table for Chlorous Acid Aqueous Solution

TABLE 1

| | | Match/Not a |
|---|---|---|
| CAAS specification | Specification Value | Match |
| Content | 4-6% | 4.1% |
| Attribute | light yellowish green to yellowish | yellowish red |
| | red | |
| Confirmation Test (1) | When 0.1 ml of aqueous potassium | Match |
| | permanganate solution (1→300) is | |
| | added to 5 ml of an aqueous | |
| | solution of the present product | |
| | (1→20), the solution turns reddish | |
| | purple. When 1 ml of sulfuric acid | |
| | (1→20) is added thereto, the | |
| | solution turns light yellow. | |
| Confirmation Test (2) | An aqueous solution of the present | Match |
| | product (1→20) has maximum | |
| | absorbance sections at wavelengths | |
| | 258-262 nm and 346-361 nm. | |
| Confirmation Test (3) | If potassium iodide starch paper is | Match |
| | dipped in the present product, the | |
| | potassium iodide starch paper | |
| | changes to a blue color and then the | |
| | color fades. | |
| Purity Test (1) | 1.0 μg/g or lower for lead | Match |
| Purity Test (2) | 1.0 μg/g or lower for Ag₂O₃ | Match |

A chlorous acid aqueous solution formulation was manufactured using this chlorous acid aqueous solution based on the following composition.

TABLE 2

| | | Blended | | Acceptable |
|---|---|---|---|---|
| | Raw material | amount | Concentration | range |
| 1 | Tap water | 258.0 g | | |
| 2 | Dipotassium | 17.0 g | 1.70% | 0.70%-13.90% |
| | hydrogen | | | |
| | phosphate | | | |
| 3 | Potassium | 5.0 g | 0.50% | 0.10%-5.60% |
| | hydroxide | | | |
| 4 | Chlorous acid | 720.0 g | 72.00% | 0.25%-75% |
| | aqueous solution | | | |
| | (pH 2.9 to 3.5) | | | |

| | | |
|---|---|---|
| Total | Chlorous acid | 1000 g |
| | 30000 ppm | |

TABLE 3

| | Chlorous acid aqueous solution |
|---|---|
| | formulation manufactured with chlorous |
| CAAS Specification | acid aqueous solution |
| Content | 3.0% |
| Attribute | Yellow |
| Confirmation Test (1) | Match |
| Confirmation Test (2) | Match |
| Confirmation Test (3) | Match |
| Purity Test (1) | Match |
| Purity Test (2) | Match |

For the “chlorous acid aqueous solution formulation manufactured with chlorous acid aqueous solution” prepared based on the preparation method described above, the concentration of “chlorous acid aqueous solution” was measured based on the “Quantification method of chlorous acid aqueous solution” described above, and buffer prepared to achieve the free chlorine concentration described in each Example (phosphate buffer comprising dipotassium hydrogen phosphate and potassium dihydrogen phosphate) was used to prepare the chlorous acid aqueous solution of each Example.

(Test for Studying Disinfecting Effect of Chlorous Acid Aqueous Solution and Sodium Hypochlorite on M. intracellulare)

There are various disinfecting/germicidal solutions used in environmental maintenance at a hospital, etc. In particular, sodium hypochlorite, acidified sodium chlorite, etc. are used as a chlorine oxide based disinfecting/germicidal solution. In general, sodium hypochlorite is widely used.

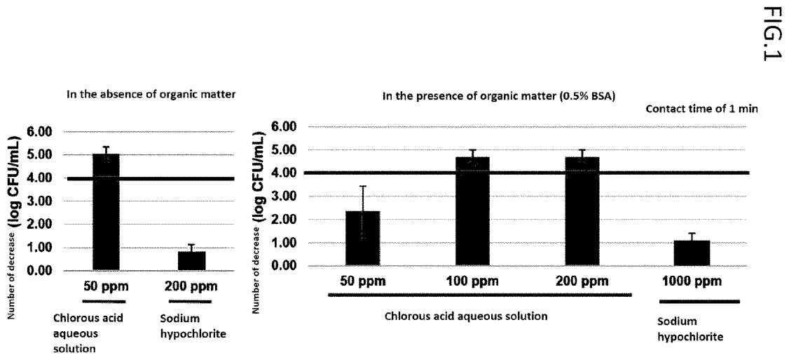

The disinfecting effects of chlorous acid aqueous solutions and sodium hypochlorite on M. intracellulare were studied in the absence of an organic matter and in the presence of an organic matter, i.e., 0.5% bovine serum albumin (hereinafter, BSA) (FIG. 1). If an effect of reducing the bacteria count is 4 log or greater, the agent is considered as having a disinfecting effect.

First, it was found that a chlorous acid aqueous solution exhibits a disinfecting effect at 50 ppm in the presence of an organic matter and at 100 ppm in the presence of 0.5% BSA, even after a short period of contact time of 1 min. In contrast, a disinfecting effect was not observed for sodium hypochlorite even at 200 ppm of free chlorine (as Cl) in the absence of an organic matter, or at 1000 ppm of free chlorine (as Cl) in the presence of 0.5% BSA.

It was found, in view of the above, that chlorous acid aqueous solutions are effective on M. intracellulare.

(Test for Studying Disinfecting Effect on M. intracellulare that Forms a Biofilm)

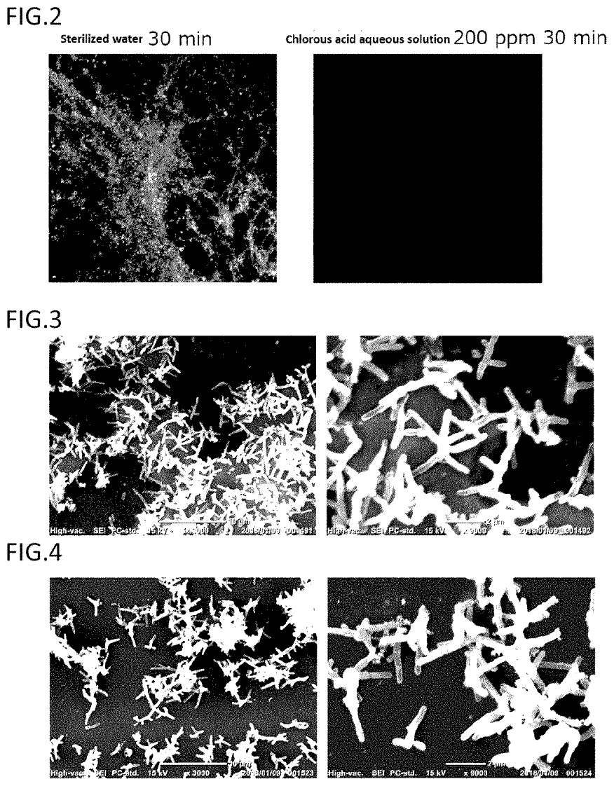

Since M. intracellulare produces a biofilm, it is understood that agents are less effective. In this regard, a test was conducted to study whether chlorous acid aqueous solutions have a disinfecting effect on such M. intracellulare that forms a biofilm (FIG. 2). FIG. 2 shows results of sterilized water for 30 minutes and chlorous acid aqueous solution at 200 ppm or greater for 30 minutes on M. intracellulare that forms a biofilm.

As a result, viable bacteria were mostly eliminated when treated with a chlorous acid aqueous solution. It was found therefrom that a chlorous acid aqueous solution exhibits a disinfecting effect on M. intracellulare in a biofilm.

(Observation of M. intracellulare Surface After Disinfection by Using a Scanning Electron Microscope (SEM))

Since it was found from the results of a test for studying a disinfecting effect that chlorous acid aqueous solutions are very effective on M. intracellulare, a test was conducted to find the principle through which bacteria are disinfected as the mechanism of the disinfecting effect.

First, bacterial cells after disinfection were observed using a scanning electron microscope in a form of comparing a chlorous acid aqueous solution and sodium hypochlorite as to how they act on the bacterial surface. When the bacteria were observed when treated with sterilized water in order to study the normal state of M. intracellulare, it was found that extracellular matrix was present on the back of the cell body (FIG. 3). Accordingly, it is understood that a biofilm is formed by such an extracellular matrix.

When treatment with sodium hypochlorite was compared with treatment with sterilized water, elimination of extracellular matrix and a slight change in the bacterial surface were observed (FIG. 4). It was found therefrom that sodium hypochlorite was not observed to have a disinfecting effect on M. intracellulare, but likely has an effect on the extracellular matrix and bacterial surface.

When treated with a chlorous acid aqueous solution, the surface remained pristine, with hardly any effect on the bacterial surface (FIG. 5). While detachment and aggregation actions on the extracellular matrix were observed, it is understood that the relationship between such detachment and aggregation actions on the extracellular matrix and disinfecting effect is low.

(Results of Pulsed-Field Gel Electrophoresis of DNA After Disinfection on M. intracellulare)

In order to study the disinfection mechanism of chlorous acid aqueous solutions, with the understanding that a chlorous acid aqueous solution is unlikely to disinfect by acting on the bacterial surface in view of the results of SEM, chromosomal DNA was extracted from bacteria after disinfection, and the state of the chromosomal DNA was examined through pulsed-field gel electrophoresis in order to study the effect of a chlorous acid aqueous solution on chromosomal DNA of bacteria.

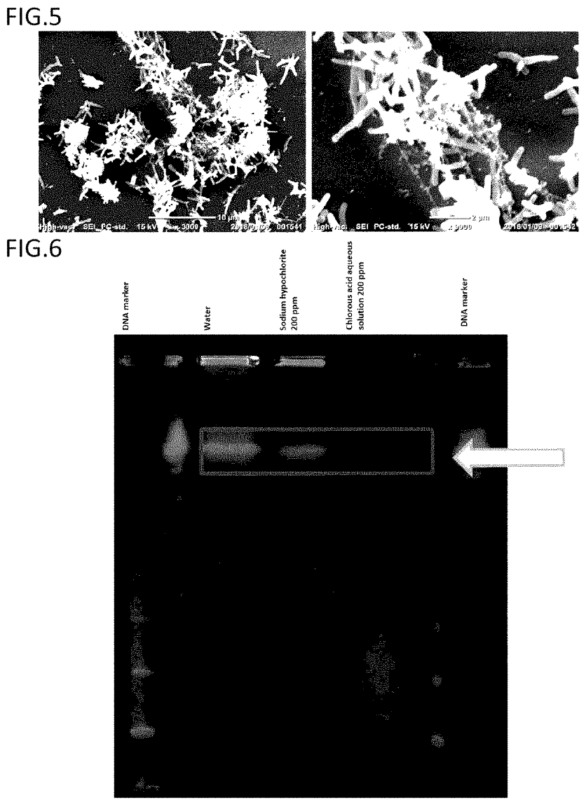

It was found as a result that a band is formed at the same position as water when treated with sodium hypochlorite, but a band is eliminated and DNA is shredded when treated with a chlorous acid aqueous solution (FIG. 6). It is understood therefrom that chlorous acid aqueous solutions infiltrate into the cells of M. intracellulare and decompose chromosomal DNA, and damage to chromosomal DNA is one of the disinfecting actions of chlorous acid aqueous solutions.

(Results of Studying the Disinfecting Effect on Other Mycobacteria)

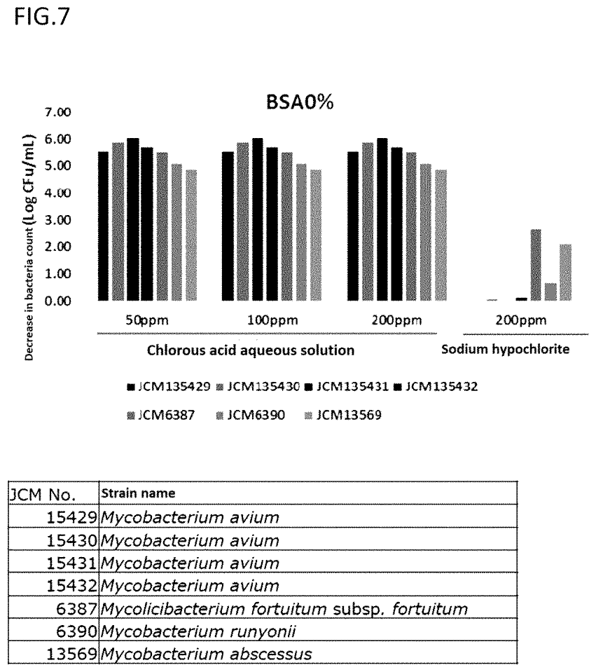

Additional studies were conducted to find whether a disinfecting effect is observed on other mycobacteria. In the absence of organic matters, an effect of chlorous acid aqueous solutions was also observed on other mycobacteria at free chlorine of 50 ppm or greater, whereas an effect was not observed from sodium hypochlorite at 200 ppm (FIG. 7). The same results as the results for M. intracellulare were obtained.

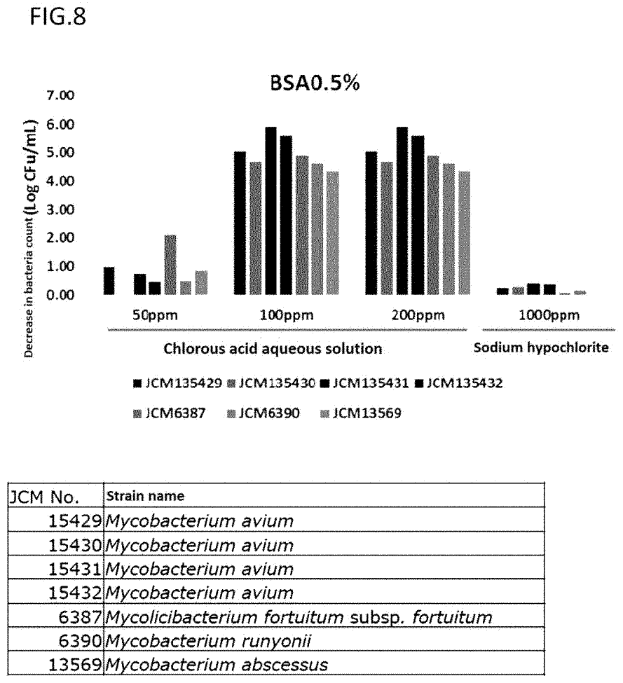

In the presence of 0.5% BSA, an effect from a chlorous acid aqueous solution was observed at free chlorine of 100 ppm or greater, whereas an effect from sodium hypochlorite was not observed even at 1000 ppm (FIG. 8). The results obtained are the same as the results for the disinfecting effect on M. intracellulare. In view of the above, a difference in the disinfecting effect of chlorous acid aqueous solutions on mycobacteria depending on the strain was not found.

### CONCLUSION

*Chlorous acid aqueous solutions exhibited a more potent disinfecting effect on M. intracellulare in comparison to sodium hypochlorite. Chlorous acid aqueous solutions also exhibited a sufficient effect on other mycobacteria.
*Chlorous acid aqueous solutions also exhibited a disinfecting effect on M. intracellulare in a biofilm.
*While disruption of the bacterial surface due to treatment with a chlorous acid aqueous solution was not observed, a potent shredding phenomenon on chromosomal DNA was observed.
*It is understood that chlorous acid aqueous solutions are less likely to be blocked by an organic matter compared to sodium hypochlorite, infiltrate into the cells, and shred bacterial molecules for disinfection.
*It is understood that DNA after disinfection is also shredded for mycobacteria other than M. intracellulare.

(Note)

As described above, the present invention is exemplified by the use of its preferred embodiments. It is understood that the scope of the present invention should be interpreted solely based on the claims. It is understood that any patent, any patent application, and any references cited herein should be incorporated herein by reference in the same manner as the contents are specifically described herein. The present application claims priority to Japanese Patent Application No. 2019-173414 (filed on Sep. 24, 2019). It is understood that the entire content thereof is incorporated herein by reference. It is understood that any patent, any patent application, and any references cited herein should be incorporated herein by reference in the same manner as the contents are specifically described herein.

### INDUSTRIAL APPLICABILITY

A method of sterilizing a mycobacterium is obtained. A method of preventing/treating MAC lung disease is obtained.

1.-9. (canceled)

10. A method of sterilizing a mycobacterium by using a chlorous acid aqueous solution.

11. The method of claim 10, wherein the mycobacterium is contacted with the chlorous acid aqueous solution in the absence of an organic matter.

12. The method of claim 11, wherein a free chlorine concentration (as Cl) of the chlorous acid aqueous solution is 50 ppm or greater and 60000 ppm or less.

13. The method of claim 10, wherein the mycobacterium is contacted with the chlorous acid aqueous solution in the presence of an organic matter.

14. The method of claim 13, wherein a free chlorine concentration (as Cl) of the chlorous acid aqueous solution is 100 ppm or greater and 60000 ppm or less.

15. A method of preventing and/or treating MAC lung disease in a subject, comprising administering an effective amount of a chlorous acid aqueous solution to the subject, or contacting an effective amount of a chlorous acid aqueous solution with a mycobacterium that causes MAC lung disease.

16.-18. (canceled)

19. A method of sterilizing a bacterium that forms a biofilm, comprising contacting an effective amount of a chlorous acid aqueous solution with the bacterium that forms a biofilm.

20. (canceled)

21. The method of claim 10, wherein the mycobacterium has formed a biofilm.

22. The method of claim 10, wherein the mycobacterium is a nontuberculous mycobacterium.

23. The method of claim 10, wherein the mycobacterium is Mycobacterium intracellulare, Mycobacterium avium, Mycolicibacterium fortuitum subsp. fortuitum, Mycobacterium runyonii, Mycobacterium abscessus, Mycobacterium kansasii, M. avium subsp. paratuberculosis, or Mycobacterium ulcerans.

24. The method of claim 15, wherein a free chlorine concentration (as Cl) of the chlorous acid aqueous solution is 50 ppm or greater and 60000 ppm or less.

25. The method of claim 15, wherein a free chlorine concentration (as Cl) of the chlorous acid aqueous solution is 100 ppm or greater and 60000 ppm or less.
